(19) United States
(12) Patent Application Publication
(10) Pub. No.: US 2019/0279166 A1
(43) Pub. Date: Sep. 12, 2019

(54) Apparatus and Method for Preventive-Maintenance Procedures Management

(71) Applicant: William Kelly MacInnis, Longmont, CO (US)

(72) Inventor: William Kelly MacInnis, Longmont, CO (US)

(21) Appl. No.: 16/297,301

(22) Filed: Mar. 8, 2019

Related U.S. Application Data
(60) Provisional application No. 62/640,688, filed on Mar. 9, 2018.

(51) Int. Cl. G06Q 10/00; G06Q 50/20; G16H 40/40; G10L 15/22; G10L 15/26; G06F 3/16

(52) U.S. Cl. CPC G06Q 10/20; G06Q 50/2057; G06F 3/167; G10L 15/22; G10L 15/265; G16H 40/40

(57) ABSTRACT

An apparatus and method that manages preventive-maintenance procedures among users and machines in a cloud network. It employs machine-readable language to: collect written documentation; convert digital audio recordings of crowd-sourced information; parse data; aggregate, normalize, compile and output the data into user-specified reports. Procedures, manuals, equipment maintenance data, and credential data go in; detailed histories of equipment maintenance and user information go out. The apparatus and method facilitates documentation of institutional-equipment-maintenance procedures at various participating locations as the maintenance is performed by a technician. It provides reports about machines in an organization, and groups of machines of identical model numbers among organizations, for use by technicians, managers and manufacturers. It serves to share the workload of managing preventive-maintenance procedures among organizations that use the data, and to provide information lifecycles of equipment.

### TECHNICAL FIELD

The present disclosure relates to apparatuses and methods that report and manage equipment-maintenance procedures.

### BACKGROUND

Preventive-maintenance procedures are sets of checks and care performed by technicians for the purpose of maintaining equipment in satisfactory operating condition. Performance of equipment-maintenance procedures is a requirement in regulated industries like aerospace, energy utility, mining, manufacturing and healthcare. In particular, hospitals employ equipment-maintenance procedures to maintain the safety of their medical equipment. Using Computerized Maintenance Management Systems (CMMS), hospitals perform, document and track routine maintenance of equipment according to healthcare-industry and government standards. Equipment owners are required to document maintenance procedures they have performed, as well as when they performed them, who performed them, and the results. The FDA closely scrutinizes medical device companies to ensure the safety of these devices.

A 2013 mandate from the Centers for Medicare and Medicaid Services (CMS) required hospital facilities, supplies and medical equipment to be maintained to an acceptable level of safety and quality. Hospitals must compile full inventories, follow manufacturer maintenance recommendations, identify high-risk equipment, and maintain policies and procedures that monitor the credentials of those who maintain the equipment. Most hospitals lack adequate tools or systems to fully comply with these regulations.

Public online databases such as Medwatch list device failures and sentinel events, but such reporting is only voluntary, and occurs only after a failure or sentinel event.

An automated system with a centralized database and evaluation loop could distribute the equipment-maintenance management burden. It could help manufacturers receive lifecycle data automatically, helping them anticipate device failures.

In most hospitals, gathering detailed data on the use of preventive maintenance procedures is a manual process with many potential failure points. Because existing systems do not adequately support these procedures, organizations may not fully comply with the strict government and industry requirements for performing them. Despite the CMS's mandate and equipment manufacturers' procedural guidelines, data collection is rudimentary. Current CMMS systems fail to document hospital procedures that align with industry recommendations. These systems effectively track equipment but inadequately gather detailed information about the maintenance performed on the equipment. CMMS systems attempt to provide a system for procedure documentation, but fail to offer reference to manufacturers' manuals or facilitate timely procedure development, or to offer indexable procedures to technicians as they perform them. Indexable procedures would further serve in procedure-data collection and post-procedure reference.

Healthcare and other industries would benefit from systems and methods that facilitate timely reporting on equipment-maintenance procedures, as well as aid in developing in-house standards for doing so.

Equipment owners are required to develop procedures and schedules for maintaining their equipment. These procedures must follow manufacturers' recommendations. Hospitals gather and store these recommendations to make them available to technicians as they perform preventive maintenance.

Medical-equipment manufacturers provide check lists, manuals and standardized procedures for proper preventive maintenance of their products by end-users. However because hospitals perform this maintenance individually (per hospital) rather than collectively (across an industry), the result is siloed data that is not cycled back to manufacturers, who might use the data for quality control. When procedures are adhered to, the resulting data remains sequestered in an organization instead of gathered and aggregated into a central database, where it could be put to use managing and distributing the work of preventive maintenance. Because preventive-maintenance procedures are complex and take time to record, industries would be better served to share that work as much as possible.

“Crowdsourcing” is the work of numerous individuals who contribute information or work to be collected, aggregated and distributed via the World Wide Web. In an example, individuals may respond to an online survey about their sleep habits. The web site hosting the survey collects, analyzes, sorts and uses the data in their research. In this case the work is done by many and collected and used for the purpose of research.

An end-user is a trained staff-person who uses equipment that requires maintenance. In the context of this embodiment an end-user may be a technician, manager or manufacturer.

A cloud server is a database that resides on a computer server that is connected to the Internet for the purpose of collecting, parsing, aggregating, normalizing, reporting and sharing information online.

A procedure history is a written report of a maintenance procedure according to type of equipment.

Voice-input is the recorded voice of an end-user who performs the equipment maintenance.

Normalization, or Database Normalization, is the process of structuring a relational database in accordance with a series of normal forms to reduce data redundancy and improve data integrity. In the context of this invention, normalization keeps data uniform, preventing duplicate data in instances of, for example, similar names, preventing misnamed objects throughout a dataset.

The instant invention is referred to herein as both “apparatus and method” and “embodiment.”

### SUMMARY

The present embodiment is an apparatus and method for collecting, parsing, aggregating, normalizing, reporting and sharing preventive-maintenance procedures. It employs machine-readable language to collect and convert cloud-sourced procedure data, in the form of both written and digital audio recordings, into data that is aggregated, normalized, stored on and shared in reports from a cloud server. The apparatus and method facilitates documentation of preventive-maintenance procedures by collecting preventive-maintenance procedure data; comparing it with and normalizing it with data from other machines at various participating institutions; aggregating the data into various types of reports by user specification; and sharing compiled data across a cloud network of participating institutions. In this way the embodiment provides an information lifecycle of institutions' equipment. The apparatus and method achieves this in four general ways: 1.) By facilitating the downloading, entering and uploading of preventive-maintenance documentation into a central database as the work is being performed on site; 2.) By facilitating for manufacturers the uploading or entering of source documentation (such as equipment manuals); 3.) By parsing and comparing data about the same machines at various institutions and normalizing it with data from other machines at various institutions, and using authoritative data sources like the FDA to populate new equipment and company records; 4.) By aggregating and compiling the data into various types of reports according to user specification and according to the type of company the user represents.

The sum of this apparatus and method is a comprehensive set of preventive-maintenance lifecycle data and histories which can be produced into uniquely specified reports, and shared among equipment owners.

The apparatus and method also gathers source data from manufacturers; facilities the editing of data by manufacturers; and stores the data in a way that can be referred to during preventive maintenance. The apparatus and method provides reports about machines in an organization, and groups of machines of identical model numbers among organizations, for preventive-maintenance-procedure management by end-users and manufacturers.

The embodiment's graphical user interfaces vary by type of user, including technician, manager, and manufacturer.

While performing preventive maintenance procedures, technicians may view, in a single window of the application's interface, both a manufacturer's manual and a window for documenting their procedure. The documentation window receives procedure data in writing or by voice input. That data is then uploaded to the embodiment's central server. Technicians may also upload their official industry certifications. Their certifications remain in their user profile and follow them across work locations, allowing per-diem technicians to log in at various institutions. The embodiment may notify technicians that their industry certifications are about to expire; or that they are no longer credentialed at a certain institution; or other concerns.

Managers review technicians' credentials and certifications, which are analyzed, validated, stored and sent as reports by the embodiment. Because the embodiment links procedure histories to each piece of equipment, managers may view data that is aggregated by, for example, machine, date, institution, procedure results or changes, or technician. Managers receive automatic notification of expiring user certifications or flagged credentials. Managers use call reports from the embodiment for purposes of overseeing technicians; reviewing procedures, and evaluating equipment data and histories.

Manufacturers upload their static user manuals to the embodiment. They examine and edit maintenance procedure documentations that were entered by technicians. They can see, from an end-user's perspective, exactly what aspects of their procedure documentation might have failed, and what the technician's comments were. Manufacturers get aggregated histories from individual history records of specific pieces of equipment from each procedure performed on that type of equipment at each institution. This combined data reveals trends and lifecycle information about equipment that would otherwise be difficult to acquire. Manufacturers also get aggregated training information about who is trained to maintain their equipment and who is training technicians at the institutions.

The apparatus and method collects, compares, checks, normalizes and aggregates data from various locations and users; transforms it into specified reports according to a user's need, and shares it across a cloud network of institutions. In normalizing equipment data the embodiment checks FDA registrations, GUDID databases, uploaded manuals, and currently published industry standards. In this way it facilitates adherence to industry recommendations and government standards for equipment maintenance; helps develop in-house standards for doing so; and provides a method for preventive-maintenance procedure reporting. It also serves as a reporting system that allows hospitals to obtain data about equipment; aggregate data on all of their equipment; and understand failure points and life-cycle information about equipment models they own.

Serving to share the workload of creating preventive-maintenance procedures among all institutions that need the data, the apparatus and method collects the procedures for new equipment and maintains and corrects existing preventive-maintenance procedures for existing equipment.

Institutions and maintenance companies get detailed history reports about individual pieces of equipment that they maintain. This data can be compiled into individual equipment reports that aggregate procedure executions on equipment over time. Institutions also get reports about procedure failure rates and longevity of all equipment of a given type in their inventory.

Manufacturers may choose to download failure and life-cycle information that will improve development of new equipment and improve part-creation for existing equipment.

The method and apparatus additionally serves to manage liability. Should an organization owning machines requiring preventive maintenance face allegations of wrongful death or other malfeasance, the apparatus and method would provide documented, time-stamped histories of preventive maintenance on the machine in question. Institutions may manage personnel by receiving alerts and training information about each user who maintains equipment at their institution.

One skilled in the art understands that this apparatus and method may be used in contexts additional to healthcare systems.

### BRIEF DESCRIPTION OF THE DRAWINGS

FIG. 1 is a diagram of an example embodiment.

FIG. 2 is a diagram showing user, equipment and site validation.

### DESCRIPTION

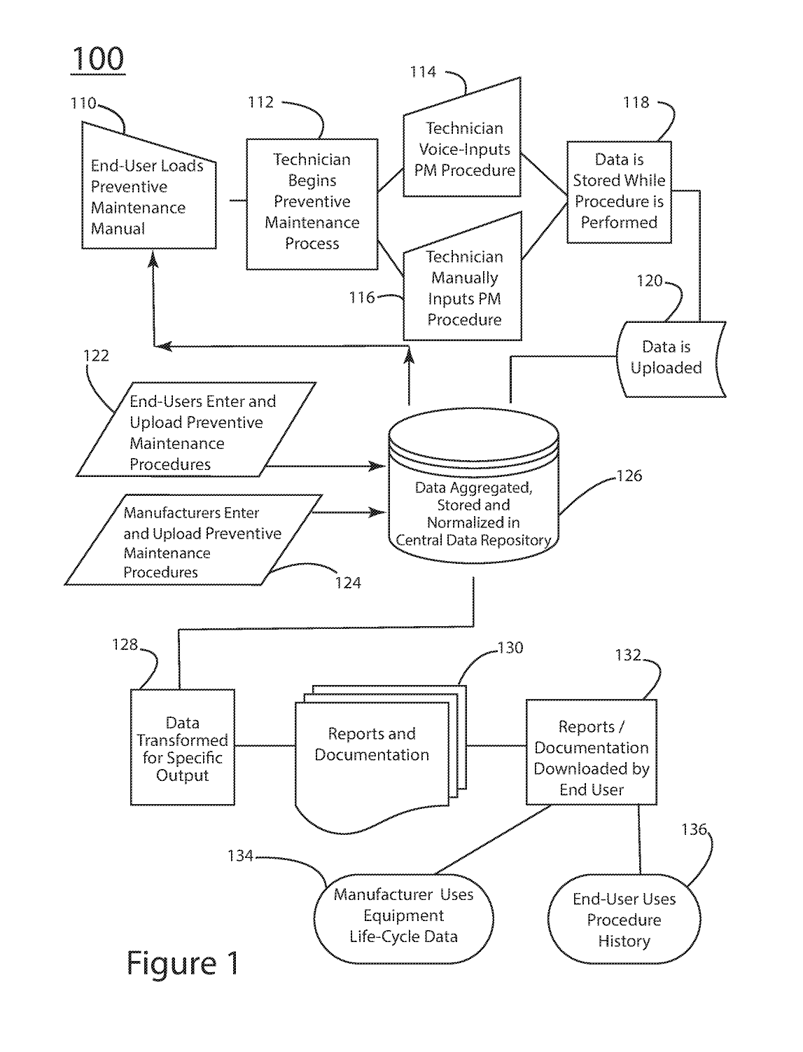

Referring to FIG. 1, in example embodiment 100:

110 End-user loads preventive-maintenance manual for specific equipment.

112 End-user begins performing preventive maintenance on the equipment, narrating the steps through voice input 114 or documenting the steps in writing 116.

118 Apparatus automatically stores data while the procedure is performed. End-users may elect to alter procedures as they perform them; the apparatus records all input.

120 Data is uploaded to the embodiment's cloud database.

122 At any time end-users upload preventive-maintenance procedures in the form of manufacturers' manuals, and/or in the form of their own amended procedures, for new equipment to the cloud database (also described as the cloud server).

124 At any time, manufacturers enter or upload their preventive-maintenance procedures manuals to the cloud database 126 (also described as the cloud server 126).

126 Uploaded data is stored in the cloud database.

128 The embodiment performs data collection, normalization, aggregation, sorting and output to specific formats. This data reports procedure results for equipment in a participating organization, as well as for groups of specified equipment models from organizations across the community of users.

130 Preventive-maintenance manuals, reports and ancillary documentation are output by user request.

132 Reports or other documentation are downloaded.

134 Manufacturers download and use manuals, reports and ancillary documentation. They may opt to view preventive-maintenance procedure reports of their equipment or detailed lifecycle data.

136 End-users and participating end-user organizations download and use manuals, reports and ancillary documentation. They may opt to view specific instances of equipment behavior or a lifecycle history. Participating manufacturers may obtain aggregate views of equipment behavior for all models and/or lifecycle data for a particular model number.

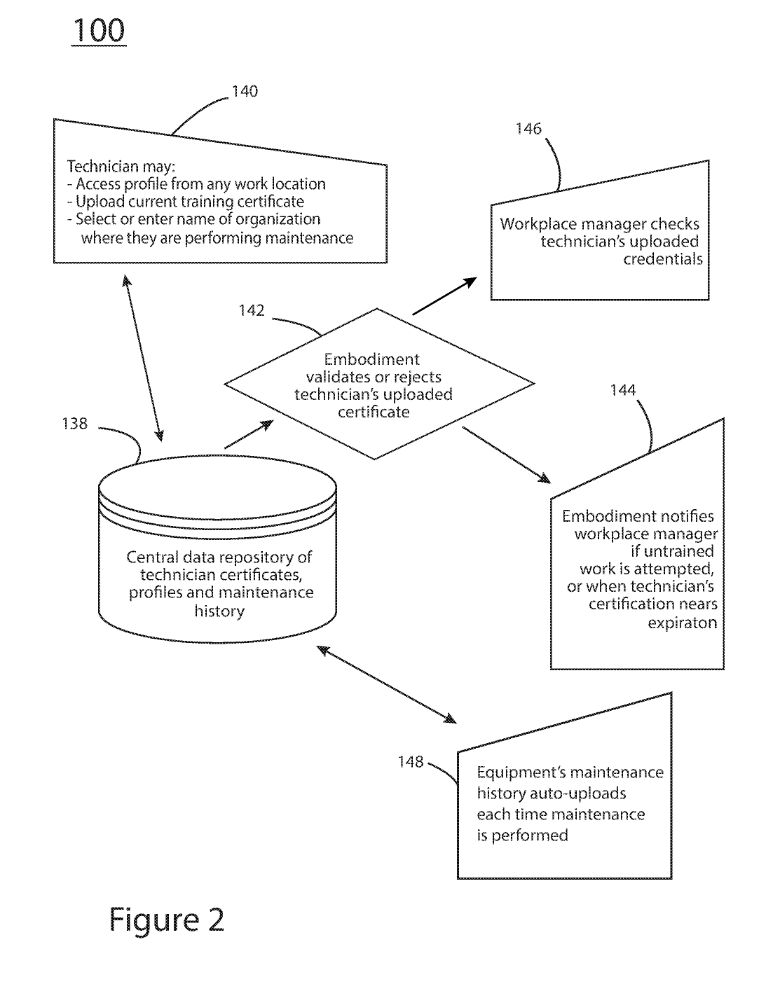

FIG. 2 shows data movement as user, equipment and sites are validated. A central data repository 138 manages technician certificates, profiles and maintenance histories which are uploaded from various participating locations.

140 A technician may access their profile from any participating work location; upload their current training certificate; and select or enter the name of the organization where they are currently performing equipment maintenance.

Once received at its central data repository 138, the embodiment 100 collects, parses, aggregates, normalizes, and compiles requested reports on preventive-maintenance procedures data.

142 The embodiment validates or rejects the technician's uploaded training certificate and sends that information to a workplace manager.

144 The embodiment notifies the workplace manager if untrained work is attempted, or when a technician's certification nears expiration.

146 A workplace manager checks the technician's uploaded credentials or any validation alerts from the embodiment.

148 The embodiment receives automatically uploaded equipment maintenance history each time maintenance is performed.

1. A computer-readable recording medium storing a data-transmission program, the data-transmission program causing a computer to perform a process comprising:
receiving preventive-maintenance manual information for a piece of equipment from a central data repository; and
receiving input data defining at least one preventive maintenance procedure, as performed by a technician; and
uploading received input data defining said at least one preventive maintenance procedure to said central data repository; and
collecting received input data, defining preventive-maintenance procedures; and
generating documentation, reporting the input data defining preventive-maintenance procedures performed by said technician; and
generating equipment-maintenance procedure history from said input data defining preventive-maintenance procedures; and
generating equipment lifecycle data from said equipment-maintenance procedure history, for dissemination to participating manufacturers, managers and technicians;
wherein
preventive-maintenance procedures as written in manufacturer's manuals, and as performed by technicians, are collected in a central data repository for the purpose of informing, updating and editing preventive-maintenance procedures which are recorded, documented and used to generate equipment-procedure history and equipment-lifecycle data, which is disseminated to participating manufacturers, managers and technicians.

2. The computer-readable recording medium storing a data-transmission program of claim 1, the data-transmission program causing a computer to perform a process further comprising:
receiving voice-input data defining said at least one preventive maintenance procedure from a technician during a preventive maintenance procedure; and
storing said voice-input data as text while said preventive-maintenance procedure is performed; and
uploading received voice-input data defining said at least one preventive-maintenance procedure to said central data repository; wherein
documentation defining preventive-maintenance procedures may be updated based on voice input from said technician while said technician is performing the preventive-maintenance procedure.

3. The computer-readable recording medium, storing a data-transmission program of claim 1, the data-transmission program causing a computer to perform a process further comprising:
receiving input data defining preventive-maintenance procedures from manufacturers; and
receiving input data defining preventive-maintenance procedures from managers;
wherein technicians, manufacturers and managers contribute to the documentation defining preventive-maintenance procedures.

4. A system for interpreting and managing preventive-maintenance data comprising:
a user interface for downloading preventive-maintenance manuals from a central data repository; and
a user interface for manual input of information derived from performing tasks defined in said preventive-maintenance manual; and
information derived from manual data entry is converted to non-transitory computer-readable medium, storing instructions; and
said instructions store preventive maintenance-manual data in a central data repository; and
said instructions store said information derived from performing tasks defined in said preventive-maintenance manual in said central data repository; and
said stored information is parsed, aggregated, normalized and compiled for output; and
said output generates preventive-maintenance procedure history; and
said preventive-maintenance procedure history is interpreted and compiled to generate equipment-lifecycle data.

5. The system for interpreting and managing preventive-maintenance data of claim 4, further comprising:
a user interface for voice-input of information derived from performing tasks defined in said preventive-maintenance manual; and
voice-input information is stored in said central data repository; wherein
documentation defining preventive-maintenance procedures may be updated based on voice-input from said technician while said technician is performing the preventive-maintenance procedure.

6. The system for interpreting and managing preventive-maintenance data of claim 4, further comprising:
a user interface for manual input of descriptions of preventive-maintenance procedures; and
manually input information is stored in said central data repository; wherein
documentation defining preventive-maintenance procedures may be updated based on manual input from said technician while said technician is performing the preventive-maintenance procedure.

7. The system for interpreting and managing preventive-maintenance data of claim 4, further comprising:
a user interface for manufacturers to input descriptions of preventive-maintenance procedures.

8. The system for interpreting and managing preventive-maintenance data of claim 4, wherein said central data repository stores manufacturer-uploaded descriptions of preventive-maintenance procedures, technician-uploaded descriptions of preventive maintenance procedures, and manager-uploaded descriptions of preventive-maintenance procedures for use by said manufacturers, technicians and managers.

9. A computer-readable recording medium storing a data-transmission program, the data-transmission program causing a computer to perform a process comprising:
receiving a user profile from a technician; and
receiving a training certificate from said technician; and
linking said training certificate to said user profile; and
receiving a maintenance event and maintenance event location, as recorded by said technician when maintenance is performed; and
storing said user profile and said linked training certificate in a central data repository; and
storing maintenance events and locations in a central data repository; and
generating documentation relating to technician-training certificates for validation; and
validating said technician-training certificates; and
generating equipment-maintenance history according to stored maintenance events;
generating a warning when a maintenance event is received without a validated technician certificate; wherein
a technician's training certificate is validated and referenced upon commencement of a maintenance event; maintenance events are compiled to generate maintenance history of a piece of equipment; and warnings are generated when a maintenance event is attempted without a validated technician certificate.

10. A computer-readable recording medium storing a data-transmission program of claim 9, the data-transmission program causing a computer to perform a process further comprising:
receiving training certificate start date and expiration date; and
storing certificate start date and expiration date in said central data repository; and
generating a notification of said expiration date prior to said expiration date; and
disseminating said notification to managers; wherein
the expiration of a certificate is sent to a manager when a maintenance event occurs.

11. A computer-readable recording medium storing a data-transmission program, the data-transmission program causing a computer to perform a process further comprising:
receiving training certificate start date and expiration date; and
storing certificate start date and expiration date in said central data repository; and
generating a warning of said expiration date after said expiration date has occurred; and
disseminating said warning to managers; wherein
said warning is sent to a manager when a maintenance event occurs after a certificate has expired.

12. A system for interpreting and managing technician user-profile and training-certificate data comprising:
a user interface for inputting a user profile; and
a user interface for inputting training certificate information to said user profile; and
uploading of said user profile with said training certificate information to a central data repository; and
validation of said uploaded training certificate information; and
interpretation of start date and end date of said uploaded training certificate information; and
correlation between a training certificate and referenced equipment; and
correlation between a training certificate and referenced procedures; and
a user interface for inputting the start of a maintenance procedure, the equipment upon which the maintenance procedure is to be performed, and the location of said equipment; and
generation of a signal when a maintenance procedure is performed on a piece of equipment during the pendency of a training certificate; and
generation of a signal when a maintenance procedure is performed on a piece of equipment when the pendency of a training certificate is near expiration; and
generation of an alert signal when a maintenance procedure is performed on a piece of equipment when a training certificate has expired; wherein
a manager receives said alert signals when maintenance procedures are performed.
